(19) United States
(12) Patent Application Publication
(10) Pub. No.: US 2026/0286836 A1
(43) Pub. Date: Sep. 24, 2026

(54) SYSTEM AND METHOD OF MONITORING SUBTERRANEAN RESERVOIRS

(71) Applicant: RESMAN AS, Ranheim (NO)

(72) Inventors: Sven Kristian HARTVIG, Ranheim (NO); Roy GREIG, Ranheim (NO); Øyvind DUGSTAD, Ranheim (NO); Olaf Kristoffer HUSEBY, Ranheim (NO)

(21) Appl. No.: 19/473,548

(22) PCT Filed: Apr. 19, 2024
(86) PCT No.: PCT/EP2024/060840

§ 371 Date: Oct. 8, 2025

(30) Foreign Application Priority Data
Apr. 19, 2023 (GB) GB2305719.3

(51) Int. Cl. E21B 47/11; F24T 10/00; F24T 10/20; G01N 33/28; G01V 20/00

(52) U.S. Cl. CPC E21B 47/11; F24T 10/20; G01N 33/2823; G01N 33/2835; G01V 20/00; F24T 2010/56

(57) ABSTRACT

The invention provides a system and method for monitoring a subterranean formation. The method comprises injecting injection fluid into the at least one injection well wherein the at least one injection well is in fluid communication with the subterranean formation. The method comprises injecting or releasing at least one tracer into the at least one injection well and taking at least one sample from fluid produced from the subterranean formation. The method comprises measuring a concentration of the at least one tracer in the at least one sample and based on the measured concentration of the at least one tracer monitoring at least one characteristic of the subterranean formation.

The present invention relates to subterranean formations and monitoring flow in subterranean formations. The invention has particular, although not exclusive, application to geothermal reservoirs and reservoirs used for carbon storage.

### BACKGROUND TO THE INVENTION

A geothermal reservoir is a naturally occurring area of hydrothermal resources. These reservoirs are deep underground and are largely undetectable above ground. A geothermal production well is drilled into a known geothermal reservoir and hot geothermal fluids flow through the production well to a power plant for use in generating electricity or the hot fluid is utilized through heat exchanger for heating or similar purposes. An injection well is drilled into the known geothermal reservoir to return used geothermal fluids to the geothermal reservoir or to introduce fluid into a dry heated rock. A geothermal system may comprise multiple injector and/or production wells.

An oil and gas reservoir is subsurface accumulation of hydrocarbons contained in porous or fractured rock formations. A production well is drilled into or through the reservoir primarily for producing oil or gas. In an oil and gas system an injection well is drilled into or through the reservoir to inject fluids into the reservoir to primarily maintain reservoir pressure to assist production into the production well or displace hydrocarbons by other fluids. An oil and gas system may comprise multiple injector and/production wells.

A carbon capture storage (CCS) reservoir is a subsurface formation used to store carbon dioxide (CO2) captured from industrial processes, or directly from the atmosphere. Captured CO2 is injected into subsurface formations such as oil, water or gas reservoirs, coal seams or salt dome reservoirs to store it permanently. Monitoring injected CO₂ in its reservoir is crucial for predicting the risk of CO₂ leakage, increasing efficiency, reducing the cost of CO₂ storage, and reducing the risk of induced seismicity.

It is known to use chemical tracers to provide a limited understanding of oil and gas reservoir fluid flow pathways for example oil companies use tracers to determine the connectivity between an injection and a production well in oil and gas reservoirs.

Tracers are labels that tag a subset of a given quantity, acting like the subset without affecting that quantity in any way. An ideal tracer for a given quantity is thus a perfect copy of elements of the set, different from the rest only in the ability to distinguish the tracer from the other elements. Molecules labelled with particular isotopes of given elements are close to ideal tracers. However, in practical applications isotopes such as tritium or deuterium are severely limited. Regulatory and other issues makes the use of radioactive isotopes difficult to handle in practice and particular isotopes can also be very costly to employ. Additionally, there are very few distinct isotopes available, hence the number of tracers based on distinct isotopes are also very few.

Tracers must be chemically and biologically stable both at all relevant conditions and behave as close as possible to the tracked fluid or phase. Suitable tracers must also be environmentally acceptable, and unique in the reservoir environment. Furthermore, tracers must be quantifiable at very small concentrations to limit injection to reasonable amounts. Finding tracer molecules that give a good representation of the fluid or phase being monitoring is challenging.

### SUMMARY OF THE INVENTION

There is a need to obtain information on mass transport and flow into, through and from subterranean reservoirs.

There is generally a need for a system and method to understand flow paths of fluid injected into a reservoir to understand its migration to, through and from the reservoir.

It is amongst the aims and objects of the invention to provide a system and method which obviates or mitigates one or more drawbacks or disadvantages of the prior art subterranean reservoir monitoring systems.

It is an object of the invention to provide a system and method to obtain information on mass transport and flow into, through and/or from geothermal reservoirs. This may allow energy production from geothermal reservoirs to be controlled and/or optimised.

It is another object of the invention to provide a system and method to obtain information on mass transport and flow into, through and/or from hydrocarbon reservoirs.

It is a further object of the invention to provide a system and method to obtain information on mass transport and flow into, through and/or from carbon capture storage reservoirs. This may allow leaks from carbon capture storage reservoirs to be detected and/or the amount of carbon dioxide permanently stored to be quantified.

It is another object of an aspect of the present invention to reliably determine the injection source of fluid produced by a reservoir.

Further aims and objects of the invention will become apparent from reading the following description.

According to a first aspect of the invention, there is provided a method for monitoring a subterranean formation, the method comprising:
injecting injection fluid into the at least one injection well, wherein the at least one injection well is in fluid communication with the subterranean formation;
injecting or releasing at least one tracer into the at least one injection well;
taking at least one sample from fluid produced from the subterranean formation;
measuring a concentration of the at least one tracer in the at least one sample;
based on measured concentration of the at least one tracer monitoring at least one characteristic of the subterranean formation.

The subterranean formation may be selected from the group comprising a geothermal reservoir, a hydrocarbon reservoir; a carbon capture storage reservoir; a carbon dioxide storage reservoir, a subterranean hydrogen storage reservoir, a water reservoir and/or a shale reservoir.

The at least one characteristic of the formation may be selected from the group comprising flow characteristics, flow paths, injection flow paths, production flow paths, transport paths, reinjection flow paths, flow direction, flow channels, layers, frack characteristics, rate of cooling, rate of heating; injection rates, production rates, reservoir residence time, retention times, fluid residence time, injection fluid temperature, produced fluid temperature, reservoir temperature, sweep volumes, reservoir connections and/or well connectivity, communication between injector and producer, breakthrough time, reservoir volume, reservoir geometry, rock surface area, rock type, minerology, mineral type, enthalpy, enthalpy output, mixing capacity of cold injected fluid with hot fluid in the reservoir, heat equilibrium rate of the reservoir, rock mechanics, pressure, gravity, density, viscosity, pressure, reservoir permeability, reservoir heterogeneities, solubility, fluid chemistry, porosity and/or fluid saturation.

The method may comprise optimising an injection configuration of the injection fluid and/or a production configuration of the produced fluid based on the measured concentration of the at least one tracer. The injection configuration may be an injection rate, injection concentration, injection location, number of injection locations, injection time, injection well location, injection location in the injection well, injection frequency and/or injection volume. The production configuration may be a production rate, production location, production time, sampling time, sampling location, production frequency and/or production volume.

The method may comprise controlling and/or optimising the rate of injection and/or rate of producing fluid from the subterranean formation. The method may comprise controlling and/or optimising the rate of injection and/or rate of producing fluid from the subterranean formation based on measured concentration of the at least one tracer. The injection fluid may be a liquid, a gas and/or a supercritical fluid. The injection fluid may be liquid, gas, water, wastewater, groundwater, brine (salt water), carbon dioxide, nitrogen or water mixed with chemicals. The injection fluid may be carbon dioxide, nitrogen and/or hydrocarbon gas.

The at least one tracer may be released or injected into the injection fluid via at least one tracer release device. The at least one release device may be at least one tracer injection device. The at least one tracer may be released or injected into the injection fluid at surface. The at least one tracer may be released or injected into the injection fluid downhole. The tracer release device may be located at surface. The at least one tracer release device may be located downhole. The at least one tracer injection device may be located at surface. The at least one tracer injection device may be located downhole. The at least one tracer injection device or at least one tracer release device may be permanently installed in a well or injection site. The method may comprise adjusting and/or controlling the position or location of the least one tracer release device.

The method may comprise adjusting and/or controlling the duration, concentration, and/or frequency of the injection or release of tracer into the injection fluid. The method may comprise controlling and/or adjusting the release of tracer into at least one injection fluid for a desired duration and/or frequency. The method may comprise actuating the tracer injection device to release tracer. The method may comprise injecting or releasing tracer continuously. The method may comprise injecting or releasing tracer continuously for a sustained period of time. The method may comprise actuating the at least one tracer release device to allow continuous release of tracer.

The method may comprise injecting or releasing tracer as a pulse. The method may comprise injecting or releasing tracer for a short period of time to form a tracer pulse. The method may comprise actuating a tracer injection device to allow pulsed release of tracer. The at least one tracer injection device may be configured to control the interval between each tracer release which is also known as the frequency of tracer release pulse. The at least one tracer injection device may be configured to control the duration and/or frequency of the tracer release pulse. The tracer release pulse duration range may be between 0.01 seconds and 100 days. The tracer release pulse duration range may be between 10 seconds and 1 month. The tracer release pulse duration range may be between 30 minutes and 10 days. The tracer release pulse duration range may be between 2 hours and 3 days. The tracer release pulse duration range may be between 30 minutes and 10 hours.

The volume, concentration, duration, release rate and/or frequency of the at least one tracer release may be adjustably set. The tracer injection device may be controlled and/or programmed to release tracer periodically for example hourly, weekly or monthly. The period may be adjustably controlled and set. The tracer release may be controlled and/or programmed to release tracer manually or automatically.

The method may comprise releasing or injecting tracer on command. The method may comprise releasing or injecting tracer in response to a timer and/or a control signal. The method may comprise releasing or injecting tracer in response to a pre-set programme and/or timer. The method may comprise releasing or injecting tracer in response to a trigger event. The at least one tracer release device may be controlled to selectively release tracer into the injection well to allow characteristics of the formation to be monitored. Flow measurement, flow paths and/or transport paths may be identified, calculated and/or monitored.

The at least one tracer release device may be configured to release at least one tracer. The at least one tracer release device may comprise a premixed tracer. The injection fluid and the at least one may be premixed before injection of the injection fluid. The injection fluid and the at least one tracer may be mixed during injection of the injection fluid. The at least one tracer source may be located in a flow path of the injection fluid. The at least one tracer may be configured to be released from at least one tracer source on command and/or on contact with the injection fluid. The at least one tracer may be configured to release 50% of the tracer from the at least one tracer source over a period of 1 hour to 10 years. The at least one tracer may be configured to release 50% of the tracer over a period of 1 hour to 5 years. The at least one tracer may be configured to release 50% of the tracer over a period of 1 day to 24 months. The at least one tracer may be configured to release 50% of the tracer over a period of between 1 day to 3 weeks. The at least one tracer may be configured to release 50% of the tracer over a period of between 1 week to 6 months. The at least one tracer may be configured to release 50% of the tracer over a period of between 1 month to 24 months.

The tracer material may comprise a tracer and a carrier. The carrier may be a matrix material. The matrix material may be a polymeric material. The tracer may be chemically immobilized within and/or to the carrier. The tracer material may be chemically immobilized configured to release tracer molecules or particles in the presence of a chemical trigger or specific fluid. The carrier may be a polymer. The tracer may be physically dispersed and/or physically encapsulated in the carrier. The tracer material may release tracer molecules into fluid by dissolution or degradation of the carrier and/or the tracer into the injection fluid. The carrier may be selected to controllable degrade on contact with the injection fluid. The carrier may be selected to degrade by hydrolysis of the carrier. The tracer and/or the carrier may be fluid specific such that the tracer molecules will be released from the tracer material as a response to a contact with a target liquid such as the injection fluid. The tracers and/or the carrier may be chemically intelligent such that tracer molecules will be released from the tracer material as a response the exposure of the tracer material to a target fluid. The tracer release device may be configured to release two or more tracers. The method may comprise releasing two or more tracers in a known combination or ratio.

The at least one tracer may be a liquid, solid or gas. The at least one tracer may be a powdered solid. The at least one tracer may be selected from the group comprising chemical, fluorescent, phosphorescent and radioactive compounds isotope, isotope signature, stable isotope and/or radioactive isotope of elements constituting a part of a tracer molecule. The at least one tracer may comprise stable or radioactive isotopes of elements constituting a part of a tracer molecule. The at least one tracer may be a perfluorinated compound. The at least one tracer may comprise chemical tracers selected from the group comprising perfluorinated hydrocarbons or perfluoroethers. The perfluorinated hydrocarbons may be selected from the group of perfluoro butane (PB), perfluoro methyl cyclopentane (PMCP), perfluoro methyl cyclohexane (PMCH). The at least one tracer may be an organofluorine compound with hydrogen replaced by fluorine. The at least one tracer may be a perfluorocarbon. The at least one tracer may be a nanoparticle. The at least one tracer may be a quantum dot. The at least one tracer may be a polyaromatic sulfonate compound. The at least one tracer may be naphthalene sulphonic acid. The at least one tracer may be an artificial tracer or a natural tracer. The at least one tracer may be a water tracer. The at least one tracer may be a tracer combination and/or a tracer combination ratio. The at least one tracer may be a unique tracer, a unique tracer combination and/or a unique tracer combination ratio.

The method may comprise injecting the at least one injection fluid into at least one injection well. The method may comprise injecting an injection fluid with a first tracer at a first injection time. The method may comprise injecting an injection fluid with a second tracer at a second injection time. The method may comprise injecting an injection fluid with a first tracer into a first injection well. The method may comprise injecting a second injection fluid with a second tracer into a second injection well. The first tracer and/or second tracer may be different tracer types. The first tracer and/or second tracer may be a combination of tracers or tracer types. The first tracer may have a different tracer combination to the second tracer. The first tracer may comprise a tracer combination comprising at least one tracer which is present in the second tracer but the first and second tracers have a different tracer type combination. The first tracer may have the same tracer combination as the second tracer but the first and second tracers comprise a different combination ratio. The at least one tracer may be a tracer combination and/or a tracer combination ratio. The first tracer may be a unique tracer, a unique tracer combination and/or a unique tracer combination ratio. The second tracer may be unique tracer, a unique tracer combination and/or a unique tracer combination ratio different to the first tracer.

The method may comprise obtaining produced fluid from at least one production well. The at least one production well may be in fluid communication with the formation.

There may be two or more tracer injection devices. If there are two or more tracer release devices then they may be configured to release tracer independently from one another or in synchrony with one another. The two or more tracer release devices may control the tracer releases such that the tracer releases overlap. The two or more tracer release devices may be arranged, located or positioned in the same injection well. The two or more tracer release devices may be arranged, located or positioned in different injection wells.

The method may comprise measuring and/or calculating a baseline of tracer in the injection fluid. The method may comprise reinjecting produced fluid into the injection well. The method may comprise taking one or more samples of the reinjection fluid. The method may comprise measuring and/or calculating a baseline of tracer in the reinjection fluid. By taking the at least one sample it is meant sampling the at least one sample from fluid produced from the subterranean formation. The method may comprise collecting samples of the produced fluid. The method may comprise collecting samples of the injection fluid. The sampling may be conducted at one or more sampling times. The sampling may be conducted downhole in a production well. The sampling may be conducted at surface. The sampling may be conducted at a location in a direction towards the surface of the production well. Samples may be collected for later analysis. The collected samples may be further analysed onsite or offsite.

The method may comprise detecting the presence of tracer in the produced fluid. The method may comprise detecting the presence of tracer in the produced fluid in real time. The method may comprise detecting the presence of tracer in the produced fluid using an online analyser. The method may comprise measuring a concentration of at least one tracer in the produced fluid. The method may comprise measuring a concentration of at least one tracer in the produced fluid in real time. The method may comprise measuring a concentration of at least one tracer in the produced fluid using an online analyser. The method may comprise detecting the presence of tracer in the injection fluid and/or re-injection fluid. The method may comprise detecting the presence of tracer in the injection fluid and/or re-injection fluid in real time. The method may comprise measuring a concentration of at least one tracer in the injection fluid and/or re-injection fluid. The method may comprise measuring a concentration of at least one tracer in the injection fluid and/or re-injection fluid in real time. The method may comprise online, inline, at-line and/or offline sampling.

The produced fluids may be sampled at a pre-determined time sequence or pre-determined profile. The method may comprise adjusting the sample volume and/or sampling time. The sampling sequence, duration and/or frequency may be modified during the sampling operation. The sampling sequence, duration and/or frequency may be modified based on measured tracer data. The sampling sequence, duration and/or frequency may be modified based on a model.

The at least one tracer may be detected and its concentration measured by sampling fluid from the production well and/or injection well. The sampling may be achieved by a sampling device or probe arranged in the flow of produced fluid and/or injection fluid. The sampling device or probe may be located downhole or at surface. The sampling may be conducted at the one or more of said sampling times. The at least one tracer may be detected by a detection device such a sensor. The detection device may facilitate real time monitoring and/or analysis of the tracer in the flow of produced fluid and/or injection fluid. The real time monitoring and/or analysis may be achieved by a detector device or probe. The detector device or probe may be arranged in the flow of produced fluid and/or injection fluid. The detector device or probe may be located downhole or at surface.

The method may comprise conducting optical monitoring for the detection and/or concentration of the at least one tracer in the produced fluid, injection fluid and/or re-injection fluid. The method may comprise determining the type of tracer. The method may comprise the measuring and/or monitoring the concentration of tracer. The method may comprise the measuring and/or monitoring the transport time of the at least one tracer. The method may comprise the measuring and/or monitoring the transport time of the at least one tracer from injection to detection in the producing fluid.

The tracer may be detected, and its concentration measured by different techniques such as optical detection, optical fibers, spectrophotometric methods, chromatographic methods or radioactivity analysis. The method may comprise analysing at least one characteristic of the tracer release, injection, injection volume, injection location, sampling time, sampling location, transport time, tracer concentration, temperature of injection fluid and/or production fluid and/or produced volume.

The method may comprise injecting or releasing at least one tracer in two or more injection wells. The method may comprise analysing the arrival of tracer concentration of each injected or released tracer in the produced fluid. The method may comprise analysing the rate of decline of the tracer concentration in the produced fluid to determine reservoir flow, flow rates and/or flow paths.

The method may comprise modelling the formation or reservoir, tracer concentration, transport time, injection flow rate and/or production rates in a model. The formation or reservoir model profile, tracer concentration, transport time, injection flow rate and/or production rate may be adjusted until calculated concentrations of model tracers compare with the measured concentrations of identified tracers to estimate formation or reservoir characteristics. The model may comprise parameters selected from the group including: the number of injection wells, temperature, temperature of fluid at each injection well, temperature of fluid produced, number of production wells, temperature of fluid at each production well, transport time, transport time of tracer-containing fluid from injection to production for one or more injection wells, transport time of tracer-containing fluid from injection to production for one or more production wells, transport time of tracer-containing fluid through the reservoir, tracer type, tracer combination, concentration of tracer, concentration of tracer as a function of time, fluid flow path from one or more injection well to one or more production wells in communication to the geothermal reservoir, injection rate, production rate, mixing capacity of cold injected fluid with hot fluid in the reservoir, heat equilibrium rate of the reservoir, rock mechanics, pressure, gravity, density, viscosity; reservoir permeability, reservoir heterogeneities, solubility, fluid chemistry, porosity, fluid saturation, injection amount, injection volumes and/or migration path of the at least one tracer in and/or through the formation.

The method may comprise updating the model based upon measured and/or calculated data. The method may comprise history matching. The method may comprise comparing historical parameter measurements to calculated data. The method may comprise adjusting one or more parameter measurements of the model until a reasonable match is achieved between the measured and calculated data. The model may simulate characteristics of the formation and/or pathways of injected fluid and at least one tracer.

The method may comprise comparing modelled tracer sample data to measured tracer sample data.

The method may comprise transferring and/or transmitting tracer data to a remote location. The method may comprise transferring and/or transmitting tracer data from the sampling location and/or analysis location to a remote location. The method may comprise transferring and/or transmitting tracer data to a cloud platform. The method may comprise transferring and/or transmitting tracer data to a data storage location or analysis location. The method may comprise transferring and/or transmitting tracer data using Wi-Fi, GSM, satellite communications, or MQTT protocols.

The method may comprise monitoring at least one characteristic of the subterranean formation based on the measured concentration of the at least one tracer. The method may comprise optimising an injection rate of the injection fluid and/or a production rate of the produced fluid based on measured concentration of the at least one tracer. The method may comprise injecting the at least one tracer at a first time period, first injection fluid injection rate and/or first production rate to obtain a first tracer data set. The method may comprise injecting the at least one tracer at a second time period, second injection fluid injection rate and/or second production rate to obtain a second tracer data set. The method may comprise comparing the first and second data sets to monitor at least one characteristic of the subterranean formation. The method may comprise identifying trends or changes in at least one characteristic of the subterranean formation based on a comparison of the first and second data sets. The method may comprise injecting the at least one tracer at a third or subsequent time period, third or subsequent injection fluid injection rate and/or third or subsequent production rate to obtain a third or subsequent tracer data set. The method may comprise comparing the first, second and/or third or subsequent data sets to monitor at least one characteristic of the subterranean formation. The method may comprise identifying trends or changes in at least one characteristic of the subterranean formation based on a comparison of the first, second and/or third or subsequent data sets. The method may comprise modelling the subterranean formation and calibrating or validating the model based on the first, second and/or third or subsequent data sets data sets.

The subterranean formation may be a geothermal reservoir. The method may comprise controlling energy output by adjusting the injection rate of the injection fluid and/or adjusting a production rate of the produced fluid.

According to a second aspect of the invention, there is provided a method for monitoring a geothermal reservoir;
the method comprising;
injecting injection fluid into at least one injection well, wherein the
at least one injection well is in fluid communication with the geothermal reservoir;
injecting or releasing at least one tracer into the at least one injection well;
taking at least one sample from fluid produced from the geothermal reservoir;
measuring a concentration of the at least one tracer in the at least one sample;
based on measured concentration of the at least one tracer monitoring at least one characteristic of the geothermal reservoir.

The injection fluid may be a liquid or a gas. The injection fluid may be liquid, gas, water, wastewater, groundwater, brine (salt water), carbon dioxide, nitrogen or water mixed with chemicals. The method may comprise adjusting and/or controlling the duration and/or frequency of the injection or release of tracer into the injection fluid. The method may comprise controlling and/or adjusting the release of tracer into at least one injection fluid for a desired duration and/or frequency. The method may comprise adjusting and/or controlling the injection of the injection fluid into the injection well. The method may comprise injecting or releasing tracer continuously. The method may comprise injecting or releasing tracer continuously for a sustained period of time. The method may comprise actuating a tracer injection device to allow continuous release of tracer. This may allow continuous monitoring of the geothermal reservoir. The method may comprise injecting or releasing tracer as a pulse. The method may comprise injecting or releasing tracer for a short period of time to form a tracer pulse. The method may comprise actuating a tracer injection device to allow pulsed release of tracer. The at least one tracer injection device may be configured to control the interval between each tracer release which is also known as the frequency of tracer release pulse. The at least one tracer injection device may be configured to control the duration and/or frequency of the tracer release pulse. The tracer release pulse duration range may be between 0.01 seconds and 100 days. The tracer release pulse duration range may be between 10 seconds and 1 month. The tracer release pulse duration range may be between 30 minutes and 10 days. The tracer release pulse duration range may be between 2 hours and 3 days. The tracer release pulse duration range may be between 30 minutes and 10 hours.

The volume, concentration, duration, release rate and/or frequency of the at least one tracer release may be adjustably set. The tracer injection device may be controlled and/or programmed to release tracer periodically for example hourly, weekly or monthly. The period may be adjustably controlled and set. The tracer release may be controlled and/or programmed to release tracer manually or automatically. The method may comprise releasing or injecting tracer on command. The method may comprise releasing or injecting tracer in response to a timer and/or a control signal. The method may comprise releasing or injecting tracer in response to a pre-set programme and/or timer. The method may comprise releasing or injecting tracer in response to a trigger event. The tracer release device may be controlled to selectively release tracer into the injection well to allow characteristics of the geothermal reservoir to be monitored. Flow measurement, flow paths and/or transport paths may be identified, calculated and/or monitored. At least one tracer release device may be configured to release at least one tracer. The at least one tracer release device may be configured to release two or more tracers. The method may comprise releasing two or more tracers in a known combination or ratio. The method may comprise releasing two or more tracers in different combinations or ratios. The at least one tracer may be a liquid, solid or gas. The at least one tracer may be a powdered solid.

The at least one tracer may be selected from the group comprising chemical, fluorescent, phosphorescent and radioactive compounds isotope, isotope signature, stable isotope and/or radioactive isotope of elements constituting a part of a tracer molecule. The at least one tracer may comprise stable or radioactive isotopes of elements constituting a part of a tracer molecule. The at least one tracer may comprise chemical tracers selected from the group comprising perfluorinated hydrocarbons or perfluoroethers. The perfluorinated hydrocarbons may be selected from the group of perfluoro butane (PB), perfluoro methyl cyclopentane (PMCP), perfluoro methyl cyclohexane (PMCH). The at least one tracer may be an artificial tracer or a natural tracer.

The method may comprise injecting the at least one injection fluid into at least one injection well. The method may comprise injecting an injection fluid with a first tracer at a first injection time. The method may comprise injecting an injection fluid with a second tracer at a second injection time. The method may comprise injecting an injection fluid with a first tracer into a first injection well. The method may comprise injecting a second injection fluid with a second tracer into a second injection well. The first tracer and/or second tracer may be different tracer types. The first tracer and/or second tracer may be a combination of tracers or tracer types. The first tracer may have a different tracer combination to the second tracer. The first tracer may comprise a tracer combination comprising at least one tracer which is present in the second tracer but the first and second tracers have a different tracer type combination. The first tracer may have the same tracer combination as the second tracer but the first and second tracers comprise a different combination ratio. The first tracer may be a tracer combination and/or a tracer combination ratio. The first tracer may be a unique tracer, a unique tracer combination and/or a unique tracer combination ratio. The second tracer may be a tracer combination and/or a tracer combination ratio. The second tracer may be unique tracer, a unique tracer combination and/or a unique tracer combination ratio different to the first tracer. The at least one tracer may be a water, oil or gas tracer. The method may comprise obtaining produced fluid from at least one production well. The at least one production well may be in fluid communication with the formation.

There may be two or more tracer release devices. If there are two or more tracer release devices then they may be configured to release of tracer independently from one another or in synchrony with one another. The two or more tracer release devices may control the tracer releases such that the tracer releases overlap. The two or more tracer release devices may be arranged, located or positioned in the same injection well. The two or more tracer release devices may be arranged, located or positioned in different injection wells. The method may comprise reinjecting produced fluid into the injection well. The method may comprise taking or sampling one or more samples of the reinjection fluid. The method may comprise measuring and/or calculating a baseline of tracer in the reinjection fluid.

The method may comprise collecting samples of the produced fluid. The sampling may be conducted at one or more sampling times. The sampling may be conducted downhole in a production well. The sampling may be conducted at surface. The sampling may be conducted at a location in a direction towards the surface of the production well. Samples may be collected for later analysis. The collected samples may be further analysed onsite or offsite.

The method may comprise measuring and/or calculating a baseline of tracer in the injection fluid. The method may comprise detecting the presence of tracer in the produced fluid. The method may comprise detecting the presence of tracer in the produced fluid in real time. The method may comprise detecting the presence of tracer and/or concentration in the produced fluid using an online analyser. The method may comprise measuring a concentration of at least one tracer in the produced fluid. The method may comprise measuring a concentration of at least one tracer in the produced fluid in real time. The method may comprise detecting the presence of tracer in the injection fluid and/or re-injection fluid. The method may comprise detecting the presence of tracer in the injection fluid and/or re-injection fluid in real time. The method may comprise measuring a concentration of at least one tracer in the injection fluid and/or re-injection fluid. The method may comprise measuring a concentration of the at least one tracer in the injection fluid and/or re-injection fluid in real time.

The at least one tracer may be detected and/or its concentration measured by sampling fluid from the production well. The sampling may be achieved by a sampling probe arranged in the flow of produced fluid. The sampling probe may be located downhole or at surface. The sampling may be conducted at the one or more of said sampling times. The at least one tracer may be detected by a detection device such a sensor. The detection device may facilitate real time monitoring and/or analysis of the tracer in the flow of produced fluid. The real time monitoring and/or analysis may be achieved by a detector probe. The detector probe may be arranged in the flow of produced fluid. The detector probe may be located downhole or at surface. The at least one probe may be a sample collection probe, a detector probe and/or a real time detector probe. The at least one probe may be configured to online, inline, at-line and/or offline sampling.

The method may comprise conducting optical monitoring for the detection and/or concentration of the at least one tracer in the produced fluid, injection fluid and/or re-injection fluid. The method may comprise determining the type of tracer. The method may comprise the measuring and/or monitoring the concentration of tracer. The method may comprise the measuring and/or monitoring the transport time of the at least one tracer. The method may comprise the measuring and/or monitoring the transport time of the at least one tracer from injection to detection in the producing fluid.

The method may comprise analysing characteristics of the tracer release, injection, injection volume, injection location, sampling time, sampling location, transport time, tracer concentration, temperature of injection fluid and/or production fluid and/or produced volume. The method may comprise optimising an injection configuration of the injection fluid and/or a production configuration of the produced fluid based on the measured concentration of the at least one tracer. The injection configuration may be an injection rate, injection concentration, injection location, number of injection locations, injection time, injection well location, injection location in the injection well, injection frequency and/or injection volume. The production configuration may be a production rate, production location, production time, sampling time, sampling location, production frequency and/or production volume. The method may comprise injecting or releasing at least one tracer in two or more injection well. The method may comprise analysing the arrival of tracer concentration of each injected tracer in the produced fluid. The method may comprise analysing the rate of decline of the tracer concentration in the produced fluid to determine reservoir flow, flow rates and/or flow paths.

The method may comprise modelling the geothermal reservoir, tracer concentration, transport time, injection flow rate and/or production rates in a model. The geothermal reservoir model profile, tracer concentration, transport time, injection flow rate and/or production rate may be adjusted until calculated concentrations of model tracers compare or substantially match with the measured concentrations of identified or produced tracers to estimate geothermal reservoir characteristics. The model may be used to optimise and/or control the flow of fluid into, through and from the geothermal reservoir for power generation. The method may comprise optimising power generation from the geothermal reservoir by adjusting and/or controlling the rate of fluid injection into the reservoir. The method may comprise optimising power generation by adjusting and/or controlling the rate of fluid produced from the reservoir. The method may comprise optimising power generation by adjusting and/or controlling the rate of pumping hot fluid out of the reservoir. By tracing the flow into, through and out from the reservoir the heating efficiency of the geothermal reservoir can be estimated or calculated. By tracing flow paths and transport times of cold fluid injected into the reservoir via one or more injection wells, the migration of fluid through the reservoir and/or the production of hot fluid via one or more production wells the heating capacity of the geothermal system may be determined.

The model may comprise parameters selected from the group including: temperature of fluid injected, the number of injection wells, temperature, temperature of fluid at each injection well, temperature of fluid produced, number of production wells, temperature of fluid at each production well, transport time, transport time of tracer-containing fluid from injection to production for one or more injection wells, transport time of tracer-containing fluid from injection to production for one or more production wells, transport time of tracer-containing fluid through the reservoir, tracer type; tracer combination, concentration of tracer, concentration of tracer as a function of time, fluid flow path from one or more injection well to one or more production wells in communication to the geothermal reservoir, injection amount, injection rate, production rate, enthalpy, enthalpy output, residence time, mixing capacity of cold injected fluid with hot fluid in the reservoir, heat equilibrium rate of the reservoir, rock mechanics, pressure, gravity, density, viscosity; reservoir permeability, reservoir heterogeneities, solubility, fluid chemistry, porosity, fluid saturation, injection volumes and/or migration path of the at least one tracer in and/or through the formation or reservoir.

The method may comprise determining a heat equilibrium rate of the geothermal well or reservoir. The rate of injected fluid may be adjusted based on the model data. The rate of produced fluid may be adjusted based on the model data. The method may comprise maximising energy recovery from a geothermal reservoir. The method may comprise calculating fluid transport, volumes, energy output and/or energy outtake from the geothermal reservoir. The method may comprise optimising and/or controlling the fluid injection flow rate at one or more injection well. The method may comprise optimising and/or controlling the fluid injection flow rate into the reservoir. The method may comprise optimising and/or controlling one or more pumps located at one or more injection well to control the fluid injection flow rate at one or more injection well. The method may comprise optimising and/or controlling the fluid injection flow rate to control the rate of cold fluid being injected into the reservoir. The method may comprise optimising and/or controlling fluid production flow rate at one or more production well. The method may comprise optimising and/or controlling the fluid production flow rate to control the rate of fluid extracted from the reservoir. The method may comprise optimising and/or controlling one or more pumps located at one or more production well to control the fluid production flow rate at one or more production well. The method may comprise optimising and/or controlling the fluid production flow rate to control the rate of heated fluid being extracted from the reservoir. The method may comprise maintaining a constant injection flow rate and/or production flow rate once a heat equilibrium rate for the reservoir has been achieved. The method may comprise maintaining a balanced injection flow rate and/or production flow rate once a heat equilibrium rate for the reservoir has been achieved.

The method may comprise balancing the injection flow rate at one or more injection wells with the production rate at one or more production wells to obtain a heat equilibrium rate for the geothermal reservoir. The method may comprise balancing the heated fluid extracted via the one or more production well with the rate of heating injected fluid via the one or more injection wells. The method may comprise monitoring the injection rates and/or production rates. The method may comprise automatically adjusting the injection rates and/or production rates to substantially maintain a heat equilibrium rate for the geothermal reservoir. The method may comprise automatically adjusting the injection rates and/or production rates to optimise the heating capacity of the geothermal reservoir. The method may comprise automatically adjusting the injection rates and/or production rates to control and/or optimise the temperature of fluid produced from the geothermal reservoir. The information obtained from the system may be displayed on a visual user interface. The information in the model data may be displayed on a visual user interface.

Embodiments of the second aspect of the invention may include one or more features of the first aspect of the invention or its embodiments, or vice versa.

According to a third aspect of the invention, there is provided a method for controlling energy output from a geothermal system comprising
injecting an injection fluid and at least one tracer into a geothermal reservoir;
analysing fluid produced from the geothermal reservoir for the concentration of the at least one tracer;
based on measured concentration of the at least one tracer adjusting an injection rate of the injection fluid and/or adjusting a production rate of the produced fluid.

The injection fluid may be a liquid or a gas. The injection fluid may be liquid, gas, water, wastewater, groundwater, brine (salt water), carbon dioxide, nitrogen or water mixed with chemicals.

The method may comprise adjusting and/or controlling the duration, concentration and/or frequency of the injection or release of tracer into the injection fluid. The method may comprise injecting tracer continuously. This may allow continuous monitoring of the geothermal reservoir. The method may comprise injecting or releasing tracer as a pulse. The method may comprise analysing characteristics of the tracer release, injection, injection volume, injection location, sampling time, sampling location, transport time, tracer concentration, temperature of injection fluid and/or production fluid and/or produced volume. The method may comprise injecting or releasing at least one tracer into at least one injection well in fluid communication with the subterranean formation. The method may comprise injecting or releasing at least one tracer in two or more injection wells. The method may comprise analysing the arrival of tracer concentration of each injected tracer in the produced fluid. The method may comprise analysing the rate of decline of the tracer concentration in the produced fluid to determine reservoir flow, flow rates and/or flow paths. The method may comprise modelling the geothermal reservoir, tracer concentration, transport time, injection flow rate and/or production rates in a model. The geothermal reservoir model profile, tracer concentration, transport time, injection flow rate and/or production rate may be adjusted until calculated concentrations of model tracers compare with the measured concentrations of identified tracers to estimate geothermal reservoir characteristics. The model may be used to optimise and/or control the flow of fluid into, through and from the geothermal reservoir for power generation. The method may comprise optimising power generation from the geothermal reservoir by adjusting and/or controlling the rate of fluid injection into the reservoir. The method may comprise optimising power generation from the geothermal reservoir by adjusting and/or controlling the rate of fluid injection into the reservoir based on the tracer data. The method may comprise optimising power generation by adjusting and/or controlling the rate of fluid produced from the reservoir. The method may comprise optimising power generation by adjusting and/or controlling the rate of pumping hot fluid out of the reservoir. The method may comprise adjusting and/or controlling the rate of fluid produced from the reservoir based on measured concentration of the at least one tracer.

By tracing the flow into, through and out from the reservoir the heating efficiency of the geothermal reservoir can be estimated or calculated. By tracing flow paths and transport times of cold fluid injected into the reservoir via one or more injection wells, the migration of fluid through the reservoir and/or the production of hot fluid via one or more production wells the heating capacity of the geothermal system may be determined.

The model may comprise parameters selected from the group including: temperature of fluid injected, the number of injection wells, temperature, temperature of fluid at each injection well, temperature of fluid produced, number of production wells, temperature of fluid at each production well, transport time, transport time of tracer-containing fluid from injection to production for one or more injection wells, transport time of tracer-containing fluid from injection to production for one or more production wells, transport time of tracer-containing fluid through the reservoir, tracer type, tracer combination, concentration of tracer, concentration of tracer as a function of time, fluid flow path from one or more injection well to one or more production wells in communication to the geothermal reservoir, injection amount, injection rate, production rate, mixing capacity of cold injected fluid with hot fluid in the reservoir, heat equilibrium rate of the reservoir, rock mechanics, pressure, gravity, density, viscosity, reservoir permeability, reservoir heterogeneities, solubility, fluid chemistry, porosity, fluid saturation, injection volumes and/or migration path of the at least one tracer in and/or through the formation and/or reservoir. The method may comprise determining a heat equilibrium rate of the geothermal well. The rate of injected fluid may be adjusted based on the model data. The rate of produced fluid may be adjusted based on the model data. The methods may comprise maximizing energy recovery from a geothermal reservoir. The method may comprise calculating fluid transport, volumes and/or energy outtake from the geothermal reservoir.

The method may comprise optimising and/or controlling the fluid injection flow rate at one or more injection well. The method may comprise optimising and/or controlling the fluid injection flow rate into the reservoir. The method may comprise optimising and/or controlling one or more pumps located at one or more injection well to control the fluid injection flow rate at one or more injection well. The method may comprise optimising and/or controlling the fluid injection flow rate to control the rate of cold fluid being injected into the reservoir. The method may comprise optimising and/or controlling fluid production flow rate at one or more production well. The method may comprise optimising and/or controlling the fluid production flow rate to control the rate of fluid extracted from the reservoir. The method may comprise optimising and/or controlling one or more pumps located at one or more production well to control the fluid production flow rate at one or more production well. The method may comprise optimising and/or controlling the fluid production flow rate to control the rate of heated fluid being extracted from the reservoir.

The method may comprise maintaining a constant injection flow rate and/or production flow rate once a heat equilibrium rate for the reservoir has been achieved. The method may comprise maintaining a balanced injection flow rate and/or production flow rate once a heat equilibrium rate for the reservoir has been achieved. The method may comprise balancing the injection flow rate at one or more injection well with the production rate at one or more production well to obtain a heat equilibrium rate for the geothermal reservoir. The method may comprise balancing the heated fluid extracted via the one or more production well with the rate of heating injected fluid via the one or more injection wells. The method may comprise monitoring the injection rates and/or production rates. The method may comprise automatically adjusting the injection rates and/or production rates to substantially maintain a heat equilibrium rate for the geothermal reservoir. The method may comprise automatically adjusting the injection rates and/or production rates to optimise the heating capacity of the geothermal reservoir. The method may comprise automatically adjusting the injection rates and/or production rates to control and/or optimise the temperature of fluid produced from the geothermal reservoir. The information obtained from the system may be displayed on a visual user interface. The information in the model data may be displayed on a visual user interface.

Embodiments of the third aspect of the invention may include one or more features of the first or second aspects of the invention or its embodiments, or vice versa.

According to a fourth aspect of the invention, there is provided a method of monitoring flow into, through and/or from a subterranean reservoir comprising the steps of:
injecting a fluid and at least one tracer into subterranean reservoir;
taking at least one sample of fluid produced from the subterranean reservoir; and
analysing the at least one sample to measure the concentration of the at least one tracer.

The subterranean formation may be selected from the group comprising a geothermal reservoir, a hydrocarbon reservoir; a carbon capture storage reservoir; a carbon dioxide storage reservoir, a subterranean hydrogen storage reservoir, a water reservoir and/or a shale reservoir. The method may comprise monitoring flow into, through and/or from the subterranean reservoir.

The method may comprise injecting the at least one injection fluid into at least one injection well. The method may comprise injecting an injection fluid with a first tracer at a first injection time. The method may comprise injecting an injection fluid with a second tracer at a second injection time. The method may comprise injecting an injection fluid into two or more injection wells. The method may comprise injecting an injection fluid with a first tracer into a first injection well. The method may comprise injecting a second injection fluid with a second tracer into a second injection well. The first tracer and/or second tracer may be different tracer types. The first tracer and/or second tracer may be a combination of tracers or tracer types. The first tracer may have a different tracer combination to the second tracer. The first tracer may comprise a tracer combination comprising at least one tracer which is present in the second tracer but the first and second tracers have a different tracer type combination. The first tracer may have the same tracer combination as the second tracer but the first and second tracers comprise a different combination ratio. The first tracer may be a unique tracer, a unique tracer combination and/or a unique tracer combination ratio. The second tracer may be unique tracer, a unique tracer combination and/or a unique tracer combination ratio different to the first tracer. The method may comprise injecting a unique tracer, a unique tracer combination and/or a unique tracer combination ratio into at least one injection well in fluid communication with the subterranean reservoir. The method may comprise measuring a concentration of the unique tracer or unique tracer combination.

Embodiments of the fourth aspect of the invention may include one or more features of the first to third aspects of the invention or their embodiments, or vice versa.

According to a fifth aspect of the invention, there is provided a system for monitoring a geothermal reservoir, the system comprising:
at least one tracer release device configured to release at least one tracer into injection fluid;
at least one pump configured to pump injection fluid and the at least one tracer into at least a portion of a geothermal reservoir;
at least one probe;
wherein the at least one probe is configured to detect the concentration of the at least one tracer in fluid produced from the geothermal reservoir.

The at least one tracer release device may be at least one tracer injection device. The at least one tracer release device may be configured to selectively release tracer into the injection fluid. The at least one probe may be a sample collection probe, a detector probe and/or a real time detector probe. The system may comprise two or more tracer release devices. The system may comprise a data transfer mechanism. The data transfer mechanism may comprise a transmission system. The data transfer mechanism may be configured to transmit tracer data from the system to a remote location. The data transfer mechanism may be configured to transmit data from the at least one probe to a remote location. The data transfer mechanism may be configured to transmit test results from the at least one probe to a cloud platform. The transmission system may be based on Wi-Fi, GSM, satellite communications, or MQTT protocols.

Embodiments of the fifth aspect of the invention may include one or more features of the first to fourth aspects of the invention or their embodiments, or vice versa.

According to a sixth aspect of the invention, there is provided a system for monitoring a subterranean formation, the system comprising:
at least one tracer release device configured to release at least one tracer into injection fluid;
at least one pump to pump injection fluid and the at least one tracer into at least a portion of a subterranean formation;
at least one probe;
wherein the at least one probe is configured to measure the concentration of the at least one tracer in fluid produced from the subterranean formation.

The at least one tracer release device may be at least one tracer injection device. The tracer release device may be configured to selectively release tracer into the injection fluid. The tracer release device may be located at surface. The tracer release device may be located downhole. The tracer release device may be an automated tracer device configured to release tracer on demand. The tracer release device may be configured to release tracer in response to a command signal, a manual command signal, an automated command signal, a pre-set programme, timer signal and/or a trigger event. The at least one probe is a sample collection probe, a detector probe and/or a real time detector probe. The subterranean formation may be selected from the group comprising a geothermal reservoir, a hydrocarbon reservoir; a carbon capture storage reservoir; a carbon dioxide storage reservoir, a subterranean hydrogen storage reservoir, a water reservoir and/or a shale reservoir. The at least one probe may be a sample collection probe, a detector probe, an online detector probe and/or a real time detector probe. Embodiments of the sixth aspect of the invention may include one or more features of the first to fifth aspects of the invention or their embodiments, or vice versa.

According to a seventh aspect of the invention, there is provided a method for monitoring a hydrocarbon reservoir;
the method comprising;
injecting injection fluid into the at least one injection well, wherein the
at least one injection well is in fluid communication with the hydrocarbon reservoir;
injecting or releasing at least one tracer into the at least one injection well;
taking at least one sample from fluid produced from the hydrocarbon reservoir;
measuring a concentration of the at least one tracer in the at least one sample;
based on measured concentration of the at least one tracer monitoring at least one characteristic of the hydrocarbon reservoir.

The injection fluid may be a liquid or a gas. The injection fluid may be liquid, gas, water, wastewater, groundwater, brine (salt water), carbon dioxide, nitrogen or water mixed with chemicals. The injection fluid may be carbon dioxide and/or nitrogen. The at least one tracer may be a water tracer. The at least one tracer may be a hydrocarbon tracer. The at least one tracer may be an oil tracer. The at least one tracer may be a gas tracer. The at least one tracer may be a unique tracer, a unique tracer combination and/or a unique tracer combination ratio. The method may comprise injecting the at least one injection fluid into at least one injection well. The method may comprise injecting an injection fluid with a first tracer at a first injection time. The method may comprise injecting an injection fluid with a second tracer at a second injection time. The method may comprise injecting an injection fluid with a first tracer into a first injection well. The method may comprise injecting a second injection fluid with a second tracer into a second injection well. The first tracer and/or second tracer may be different tracer types. The first tracer and/or second tracer may be a combination of tracers or tracer types. The first tracer may have a different tracer combination to the second tracer. The first tracer may comprise a tracer combination comprising at least one tracer which is present in the second tracer but the first and second tracers have a different tracer type combination. The first tracer may have the same tracer combination as the second tracer but the first and second tracers comprise a different combination ratio. The first tracer may be a unique tracer, a unique tracer combination and/or a unique tracer combination ratio. The second tracer may be unique tracer, a unique tracer combination and/or a unique tracer combination ratio different to the first tracer.

Embodiments of the seventh aspect of the invention may include one or more features of the first to sixth aspects of the invention or their embodiments, or vice versa.

According to an eighth aspect of the invention, there is provided a method for monitoring a carbon capture storage reservoir; the method comprising;
injecting an injection fluid and at least one tracer into an injection well in fluid communication with the reservoir;
taking at least one sample from fluid produced from the reservoir;
measuring a concentration of the at least one tracer in the at least one sample;
based on measured concentration of the at least one tracer monitoring at least one characteristic of the reservoir.

The method may comprise establishing a carbon quota offset value based on the amount of carbon dioxide remaining in the storage reservoir. The method may comprise establishing a carbon quota offset value based on the amount of carbon dioxide released from the storage reservoir. The injection fluid may be carbon dioxide. The method may comprise quantifying the amount of injected carbon dioxide remaining in the reservoir. The method may comprise quantifying the amount of injected carbon dioxide remaining in the storage formation based on the measured tracer concentration. The method may comprise quantifying the amount of carbon dioxide leaking from the reservoir based on the measured tracer concentration. The method may comprise creating a model to simulate characteristics of the formation and/or pathways of injected carbon dioxide. The model may comprise parameters selected from the group comprising rock mechanics, temperature, pressure, gravity, density, viscosity; reservoir permeability, reservoir heterogeneities, solubility, fluid chemistry, porosity, fluid saturation, physical behaviour of carbon dioxide, tracer and/or carbon dioxide injection amounts, injection volumes, injection rates, cap rock characteristics, migration path of the tracer and carbon dioxide in and/or through the storage formation and/or chemical behaviour of carbon dioxide. The method may comprise updating the model based upon measured and/or calculated data. The method may comprise history matching. The method may comprise comparing historical parameter measurements to calculated data. The method may comprise adjusting one or more parameter measurements of the model until a reasonable match is achieved between the measured and calculated data. The model may simulate characteristics of the reservoir and/or pathways of injected carbon dioxide and at least one tracer. The method may comprise comparing modelled tracer sample data to measured tracer sample data. The reservoir may be an underground storage formation, a subsurface reservoir, an oil and/or gas reservoir, a saline formation, an abandoned coal seam, an organic-rich shale and/or a basalt formation. The method may comprise identifying the source of a leak of carbon dioxide stored in a storage reservoir based on the characteristics of the at least one tracer in the at least one sample.

Embodiments of the eighth aspect of the invention may include one or more features of the first to seventh aspects of the invention or their embodiments, or vice versa.

According to a ninth aspect of the invention, there is provided a method for monitoring a subterranean hydrogen storage reservoir;
the method comprising;
injecting an injection fluid and at least one tracer into the reservoir;
taking at least one sample from fluid produced from the reservoir;
measuring a concentration of the at least one tracer in the at least one sample;
based on measured concentration of the at least one tracer monitoring at least one characteristic of the reservoir.

The injection fluid may be hydrogen. The method may comprise quantifying the amount of injected hydrogen remaining in the reservoir. The method may comprise quantifying the amount of injected hydrogen remaining in the storage formation based on the measured tracer concentration. The method may comprise quantifying the amount of hydrogen leaking from the reservoir based on the measured tracer concentration.

Embodiments of the ninth aspect of the invention may include one or more features of the first to eighth aspects of the invention or their embodiments, or vice versa.

According to a tenth aspect of the invention, there is provided a method for monitoring a subterranean formation, the method comprising:
providing measured concentrations and type of tracer material data from at least one sample collected from a fluid produced from a subterranean formation; and
based on said concentrations of the at least one tracer monitoring at least one characteristic of the subterranean formation.

The subterranean formation may be selected from the group comprising a geothermal reservoir, a hydrocarbon reservoir; a carbon capture storage reservoir; a carbon dioxide storage reservoir, a subterranean hydrogen storage reservoir, a water reservoir and/or a shale reservoir.

Embodiments of the tenth aspect of the invention may include one or more features of the first to ninth aspects of the invention or their embodiments, or vice versa.

According to an eleventh aspect of the invention, there is provided a method for monitoring a geothermal reservoir;
the method comprising;
injecting injection fluid into at least one injection well, wherein the
at least one injection well is in fluid communication with the geothermal reservoir;
injecting or releasing at least one tracer into the at least one injection well;
sampling at least one sample of fluid produced from the geothermal reservoir;
measuring a concentration of the at least one tracer in the at least one sample;
based on measured concentration of the at least one tracer monitoring at least one characteristic of the geothermal reservoir.

The method may comprise injecting the injection fluid and at least one tracer into an injection well in response to a command signal, a manual command signal, an automated command signal, a pre-set programme, timer signal and/or a trigger event. The method may comprise releasing or dosing tracer into the injection fluid in response to a command signal, a manual command signal, an automated command signal, a pre-set programme, timer signal and/or trigger event. The injection fluid and the at least one tracer may be mixed before and/or during injection into the injection well. The at least one tracer may be injected into the injection fluid or flow of injection fluid. The method may comprise adjusting and/or controlling a duration, concentration and/or frequency of an injection or release of tracer into the injection fluid. The method may comprise injecting or releasing tracer continuously or as a pulse.

The sampling of the at least one sample of fluid may be inline, online, at-line and/or offline sampling. The method may comprise measuring a concentration of at least one tracer in the produced fluid in real time. The sampling may comprise collecting one or more samples for later analysis. The method may comprise optimising and/or controlling the flow of fluid into, through and from the geothermal reservoir. The method may comprise determining a heat equilibrium rate of the geothermal reservoir. The method may comprise maximising energy recovery from a geothermal reservoir based on the tracer data. The method may comprise controlling and/or optimising the rate of injection and/or rate of producing fluid from the geothermal reservoir based on measured concentration of the at least one tracer. The method may comprise calculating fluid transport, volumes, a heat equilibrium rate and/or energy output from the geothermal reservoir. The method may comprise modelling the geothermal reservoir, tracer concentration, transport time, injection flow rate and/or production rates in a model. The method may comprise injecting injection fluid with a first tracer at a first injection time and injecting injection fluid with a second tracer at a second injection time. The method may comprise injecting injection fluid with a first tracer into a first injection well and injecting injection fluid with a second tracer into a second injection well. The method may comprise releasing two or more tracers independently from one another or in synchrony with one another. The at least one tracer may be a unique tracer, a unique tracer combination and/or a unique tracer combination ratio. The method may comprise reinjecting produced fluid into the injection well and measuring and/or calculating a baseline of tracer in the reinjection fluid.

The method may be a computer-implemented method. The method may be a computer-implemented history matching method. The method may comprise storing the measurement data to a database. The method may comprise storing the model data to a database. The database may be a computer database.

Embodiments of the eleventh aspect of the invention may include one or more features of the first to tenth aspects of the invention or their embodiments, or vice versa.

According to a twelfth aspect of the invention, there is provided a method of collecting samples for analysis in monitoring a subterranean formation; wherein the subterranean formation comprises injected injection fluid and at least one tracer, the method comprising:
producing fluid from the subterranean formation,
collecting at least one sample from the produced fluid.

The subterranean formation may be selected from the group comprising a geothermal reservoir, a hydrocarbon reservoir; a carbon capture storage reservoir; a carbon dioxide storage reservoir, a subterranean hydrogen storage reservoir, a water reservoir and/or a shale reservoir.

Embodiments of the twelfth aspect of the invention may include one or more features of any of the first to eleventh aspects of the invention or their embodiments, or vice versa.

According to a thirteenth aspect of the invention, there is provided a method for monitoring a subterranean formation;
wherein the subterranean formation comprises injected fluid and at least one tracer, the method comprising:
analysing collected samples for the concentration of the at least one tracer;
based on measured concentration of the at least one tracer monitoring at least one characteristic of the subterranean formation.

The subterranean formation may be selected from the group comprising a geothermal reservoir, a hydrocarbon reservoir; a carbon capture storage reservoir; a carbon dioxide storage reservoir, a subterranean hydrogen storage reservoir, a water reservoir and/or a shale reservoir.

Embodiments of the thirteenth aspect of the invention may include one or more features of any of the first to twelfth aspects of the invention or their embodiments, or vice versa.

According to a fourteenth aspect of the invention there is provided a method of monitoring a subterranean formation, wherein the subterranean formation comprises at least one tracer and injection fluid;
the method comprising the steps of:
analysing measured concentration of the at least one tracer, the concentration of tracer having been measured from samples collected from produced fluid from the subterranean formation;
based on measured concentration of the at least one tracer monitoring at least one characteristic of the subterranean formation.

The subterranean formation may be selected from the group comprising a geothermal reservoir, a hydrocarbon reservoir; a carbon capture storage reservoir; a carbon dioxide storage reservoir, a subterranean hydrogen storage reservoir, a water reservoir and/or a shale reservoir.

Embodiments of the fourteenth aspect of the invention may include one or more features of any of the first to thirteenth aspects of the invention or their embodiments, or vice versa.

According to a fifteenth aspect of the invention there is provided an interpretation method for monitoring a subterranean formation wherein the at least one tracer and injection fluid was previously injected into the subterranean formation, the method comprising;
analysing tracer data previously obtained from samples collected from produced fluid from the subterranean formation;
analysing the tracer concentration to monitor at least one characteristic of the subterranean formation.

The subterranean formation may be selected from the group comprising a geothermal reservoir, a hydrocarbon reservoir; a carbon capture storage reservoir; a carbon dioxide storage reservoir, a subterranean hydrogen storage reservoir, a water reservoir and/or a shale reservoir.

Embodiments of the fifteenth aspect of the invention may include one or more features of any of the first to fourteenth aspects of the invention or their embodiments, or vice versa.

According to a sixteenth aspect of the invention, there is provided a method of monitoring a subterranean formation, the method comprising:
injecting at least one injection fluid into the at least one injection well, wherein the
at least one injection well is in fluid communication with the subterranean formation;
injecting or releasing at least one tracer into at least one injection well;
sampling at least one sample of fluid produced from the subterranean formation;
measuring a concentration of the at least one tracer in the at least one sample;
based on measured concentration of the at least one tracer optimising an injection configuration of the injection fluid and/or a production configuration of the produced fluid.

The injection configuration may be selected from the group comprising an injection rate, injection concentration, injection location, number of injection locations, injection time, injection well location, injection location in the injection well, injection frequency and/or injection volume. The production configuration may be selected from the group comprising a production rate, production location, production time, sampling time, sampling location, production frequency and/or production volume.

The subterranean formation may be selected from the group comprising a geothermal reservoir, a hydrocarbon reservoir, a carbon capture storage reservoir, a carbon dioxide storage reservoir, a subterranean hydrogen storage reservoir, a water reservoir and/or a shale reservoir. The method may comprise monitoring at least one characteristic of the subterranean formation based on the measured concentration of the at least one tracer.

The method may comprise injecting or releasing the at least one tracer at a first time period, first injection fluid injection rate and/or a first production rate to obtain a first tracer data set. The method may comprise injecting or releasing the at least one tracer at a first time period, injecting at a first injection fluid injection rate and/or producing at a first production rate to obtain a first tracer data set. The method may comprise injecting or releasing the at least one tracer at a first time period to obtain a first tracer data set. The method may comprise injecting at a first injection fluid injection rate to obtain a first tracer data set. The method may comprise producing at a first production rate to obtain a first tracer data set. The method may comprise injecting the at least one tracer at a second time period, second injection fluid injection rate and/or second production rate to obtain a second tracer data set. The method may comprise injecting or releasing the at least one tracer at a second time period, injecting at a second injection fluid injection rate and/or producing at a second production rate to obtain a second tracer data set. The method may comprise injecting or releasing the at least one tracer at a second time period to obtain a second tracer data set. The method may comprise injecting at a second injection fluid injection rate to obtain a second tracer data set. The method may comprise producing at a second production rate to obtain a second tracer data set. The method may comprise comparing the first and second data sets to monitor at least one characteristic of the subterranean formation. The method may comprise identifying trends or changes in at least one characteristic of the subterranean formation based on a comparison of the first and second data sets.

The method may comprise injecting the at least one tracer at a third or subsequent time period, third or subsequent injection fluid injection rate and/or third or subsequent production rate to obtain a third or subsequent tracer data set. The method may comprise injecting or releasing the at least one tracer at a third or subsequent time period, injecting at a third or subsequent injection fluid injection rate and/or producing at a third or subsequent production rate to obtain a third or subsequent tracer data set. The method may comprise comparing the first, second and/or third or subsequent data sets to monitor at least one characteristic of the subterranean formation. The method may comprise identifying trends or changes in at least one characteristic of the subterranean formation based on a comparison of the first, second and/or third or subsequent data sets. The method may comprise modelling the subterranean formation and calibrating or validating the model based on the first, second and/or third or subsequent data sets data sets.

The method may comprise injecting and/or releasing tracer into the injection fluid in response to a command signal, a manual command signal, an automated command signal, a pre-set programme, timer signal and/or trigger event. The injection fluid and the at least one tracer may be mixed before and/or during injection into the injection well. The method may comprise adjusting and/or controlling a duration, concentration and/or frequency of an injection or release of tracer into the injection fluid. The method may comprise injecting and/or releasing tracer continuously or as a pulse. The sampling of the at least one sample of fluid may be inline, online, at-line and/or offline sampling. The method may comprise measuring a concentration of the at least one tracer in the produced fluid in real time. The method may comprise collecting the at least one sample for later analysis. The method may comprise modelling the tracer concentration, transport time, volume, injection flow rate and/or production rate in a model. The method may comprise optimising and/or controlling the volume and/or rate of flow of fluid into, through and from the subterranean formation. The method may comprise controlling and/or optimising the rate of injection to, and/or rate of producing fluid from, the subterranean formation based on measured concentration of the at least one tracer. The method may comprise injecting injection fluid with a first tracer into a first injection well and injecting injection fluid with a second tracer into a second injection well. The method may comprise releasing two or more tracers independently from one another or in synchrony with one another. The first tracer may be a tracer combination and/or a tracer combination ratio. The at least one tracer is a unique tracer, a unique tracer combination and/or a unique tracer combination ratio.

The method may comprise reinjecting produced fluid into the injection well and measuring and/or calculating a baseline of tracer in the reinjection fluid. The subterranean formation may be a geothermal reservoir. The method may comprise controlling energy output by adjusting the injection rate of the injection fluid and/or adjusting a production rate of the produced fluid.

Embodiments of the sixteenth aspect of the invention may include one or more features of any of the first to fifteenth aspects of the invention or their embodiments, or vice versa.

According to a seventeenth aspect of the invention, there is provided an injection monitoring system for a subterranean formation, the system comprising:
at least one tracer release device configured to release at least one tracer into an injection fluid;
at least one pump configured to pump injection fluid and the at least one tracer into at least
a portion of the subterranean formation;
at least one probe;
wherein the at least one probe is configured to measure the concentration of the at least one tracer in fluid produced from the subterranean formation.

The system may facilitate optimisation or continuous improvement of the injection rates and/or injection volumes. Based on measured concentration of the at least one tracer an injection configuration of the injection fluid and/or a production configuration of the produced fluid may be optimised. This may provide an understanding of the performance of the total subterranean formation which may not be possible using a single tracer injection operation. The at least one tracer release device may be a tracer injection device. The at least one tracer release device may be configured to inject or release at least one tracer into the injection fluid of an injection well in response to a command signal, a manual command signal, an automated command signal, a pre-set programme, timer signal and/or a trigger event. The at least one tracer release device may be located at surface. The at least one tracer release device may be located downhole. The system may comprise two or more tracer release devices. The two or more tracer release devices may be arranged, located or positioned in the same injection well. The two or more tracer release devices may be arranged, located or positioned in different injection wells.

The at least one probe may be a sample collection probe, a detector probe and/or a real time detector probe. The at least one probe may be configured for inline, online at-line and/or offline sampling. The at least one probe may be located at surface. The at least one probe may be located in a production well. The at least one probe may be located downhole.

Based on the concentration of the at least one tracer in fluid produced from the geothermal reservoir changes in the performance or characteristics of the subterranean formation may be detected.

Embodiments of the seventeenth aspect of the invention may include one or more features of any of the first to sixteenth aspects of the invention or their embodiments, or vice versa.

### BRIEF DESCRIPTION OF THE DRAWINGS

There will now be described, by way of example only, various embodiments of the invention with reference to the drawings, of which:

FIG. 1 is a simplified section of a geothermal system in accordance with an aspect of the invention; and

FIG. 2 is a simplified section of a geothermal system in accordance with another aspect of the invention with three injection wells;

FIG. 3 is a flow chart showing steps for the optimization of a model of a geothermal reservoir in accordance with an aspect of the invention; and

FIG. 4 is an example graph of residence time plotted against enthalpy output (KJ/KG) for a geothermal reservoir.

### DETAILED DESCRIPTION OF PREFERRED EMBODIMENTS

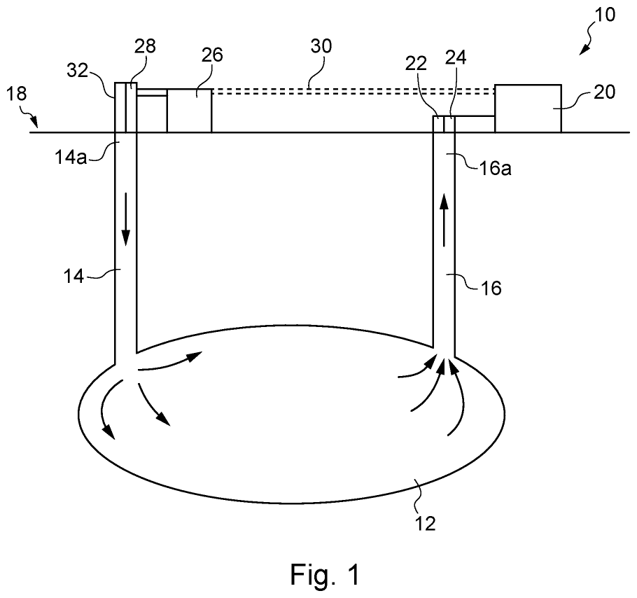

FIG. 1 is a simplified section of a geothermal system according to the invention shown generally as 10. The geothermal system has a geothermal reservoir 12 in communication with an injection well 14 and a production well 16. In this example the geothermal reservoir is in communication with one injection well and one production well. However, it will be appreciated that the geothermal reservoir may be in communication with multiple injection wells and/or multiple production well. The permeability of the geothermal reservoir may be naturally occurring or it may be enhanced by techniques such as hydraulic fracturing, pressure shearing, thermal shearing, explosive charges and/or acidification. Although FIG. 1 shows the injection well and production as a single injection and production wells. It will be appreciated that the injection well may be a network of injection wells extending from the surface 18 to the geothermal reservoir and/or the production well may be a network of production wells extending from the surface 18 to the geothermal reservoir. The geothermal reservoir may be a geothermally heated formation. The geothermal reservoir comprises a geothermal fluid disposed within the geothermal reservoir. The geothermal fluid flows within the geothermal reservoir.

In this example, an upper end 16a of the production well is in fluid communication with a power plant 20 where heated geothermal fluid is converted into electricity. A production pump 22 and sampling device 24 are located at an upper end of the production well. The production pump is configured to pump fluid from the geothermal reservoir to the power plant and the sampling device is used to measure the concentration of tracer present in samples of geothermal fluid. Replacement fluid may be stored in tanks 26 at surface and pumped via an injection pump 28 into the injection well to replace fluid in the geothermal reservoir removed from via the production well. If produced fluid is to be reinjected after it is processed through the power plant, it is returned to the storage tanks 26 via line 30. A tracer injection device 32 is located at an upper end 14a of the injection well. It will be appreciated that the tracer injection device may be located at surface or downhole. It will be appreciated that two more tracer injection devices may be located at surface or downhole at the same or different wells. The injection device is used to control the amount of tracer injected into the injection fluid in the injection well and the duration and/or frequency the tracer is injected into the injection well. Optionally if the injection fluid has been recycled i.e., reinjected fluid, it may be sampled and/or analysed for tracer to ensure a well-defined level of tracer in the fluid is injected into the injection well. As shown in FIG. 1, in use the geothermal operation follows a cycle denoted by the arrows in FIG. 1. In this example tracer is pre-mixed and loaded in the tracer injection device. The control system controls the amount of tracer injected into the injection fluid and the duration of tracer injected. In this example tracer is continuously injected into the fluid as it is pumped into the injector well.

It will be appreciated that alternative tracer release methods and mechanisms may be used to release tracer into the injection fluid. As an example, tracer source may be connected to or encapsulated by a carrier. The tracer source may be located in a flow path of the injection fluid and may be configured to selectively release tracer from the carrier on contact with the injection fluid. The tracer and injection fluid are injected using controllable pumps capable of operating at the pressure of the injection line. In this example the pump is connected to a flowline. It will be appreciated that it may alternatively be connected to a wellhead or other suitable injection point using a hose with suitable pressure rating. Typically, pneumatic pumps or electrical pumps are used for injection, but other pumping methods including manually operated pumps may be used. The tracer injection device may comprise a tracer injection pump configured to operate at a desired injection rate suitable to achieve the desired tracer concentration in the injection fluid and/or to efficiently inject the tracer in a short time period. Pumps must be rigged up ensuring integrity of the wellhead or flowline in case of hose or pump failure by e.g., installing check valves on the connection to the injection point. The release of tracer into the injection fluid may be manual, automated and/or remotely operated.

The injection fluid migrates through the geothermal reservoir, as the fluid passes through the geothermal reservoir it is heated via a heat exchange with the subterranean formation of the geothermal reservoir. Heated fluid is pumped up the production well from the reservoir to the power plant where the heated fluid is converted to electrical power. The sampling device is configured to take samples to determine the presence and concentration of the tracer in the produced fluid. In this example the sampling device is an inline sampling device and tracer is detected in real time. It will be appreciated that the sampling device may additionally or alternatively be an online, atline or offline sampling device. It will be appreciated that the sampling device may be probe such as a sample collection probe, a detector probe and/or a real time detector probe. Once the fluid has passed through the power plant it may be returned to the storage tanks. In this example a chemical tracer is used. However other tracer types including but not limited to fluorescent, phosphorescent, and radioactive compounds may be used. The tracer may comprise fluid molecules containing an isotope composition that distinguish them from fluid from other sources. In this example the fluid may be water, steam, vapour and/or a combination of any of water, steam and vapour. The tracer in this example is a water tracer, specifically a naphthalene sulphonic acid tracer.

Tracers may be analyzed using a variety of analysis techniques. Important factors will be dilution of tracers in the reservoir and number of tracers needed in a specific application. High dilution will require a lower limit of detection (LOD) for the tracer molecules. Low LOD may be obtained by e.g., detection based on fluorescence, inductively coupled plasma mass spectrometry (ICP-MS), mass spectroscopy (MS) or multidimensional MS or other highly sensitive detection methods. Analysis of many different tracers at the same time may be obtained by separation through a chromatographic column before entering a final detector. Additionally, or alternatively an isotope composition or isotopic signature of the fluid may additionally or alternatively be used as a tracer. In this example a chemical tracer is used to label the fluid. However, it will be appreciated that the tracer may alternatively be tagged, mixed, infused or co-injecting with the fluid. In this example, samples are taken continuously. The analysis of the samples in this example are carried out using a HPLC combined with a fluorescent detector. Additionally, or alternatively analysis techniques may be based on chromatographic separation followed by mass spectroscopic methods allowing quantification of tracer concentrations down to parts per trillion level. The detection of the tracer type, the concentration of tracer, injection rate, production rate and/or the transport time may be used for characterising the reservoir. The detection of the tracer type, the concentration of tracer, injection rate, production rate and/or the transport time may be used to determine flow paths and/or mass transport in the reservoir. A model of the reservoir and/or flow through the reservoir may be established.

By monitoring the transport time and the amount of tracer produced at the production well may allow a model of the reservoir to be created. The transport time depends on how much water is moving in each direction through the reservoir. The amount or portion of the injected tracer which is produced in a specific production well provides information on how much of the injected fluid has been produced at a particular production well. The measured tracer concentration in the produced samples may be assessed using interpretation method such as residence time distribution. Monitoring the transport time may provide information on the heating efficiency, volume and/or enthalpy of the geothermal reservoir. To monitor changes over time a new tracer injection can be manually or automatically carried out and the changes in flow through the reservoir monitored. To ensure control of tracers and accuracy of results, the injection fluid may be analysed to measure background of tracer in the injected fluids. To increase the energy output from the geothermal reservoir, the injection and production rates may be balanced. A model of the system may be determined and adjusted to optimise energy output. A model of the system may be determined to determine a good understanding of fluid transport within, to and/or from the reservoir. The system combines automatic injection and sample analysis of tracers with the use of data interpretation tool to calculate fluid transport, volumes, energy output and/or energy outtake from the reservoir.

The above example describes modelling of one tracer, one injection point and one sampling point. It will be appreciated that the model may be adjusted for multiple tracers, multiple injection points and/or multiple sampling points. It will be appreciated that the operational steps described above in relation to FIG. 1 are examples of the invention and that one or more steps may be omitted or added and/or that the sequence of the steps may be different and/or steps may overlap in time.

It will be appreciated that the model of the geothermal reservoir and/or pathways of injected fluid may include parameters including rock mechanics, temperature, pressure, gravity, density, viscosity; reservoir permeability, reservoir heterogeneities, solubility, fluid chemistry, porosity, fluid saturation, physical behaviour of geothermal fluid and/or chemical behaviour of geothermal fluid. Interpretation and/or modelling of tracer data may assess residence time distribution analysis to assess several characteristic properties of the flow in the geothermal system by means of tracers. Residence time distribution is a distribution of times used by a population of tracer particles to travel through a medium. The tracers represent elements of fluid that travel through different paths, and that therefore use different amounts of time to pass through a medium. The distribution, E(t), of these times is called residence time distribution, of the fluid in the system.

E(t) is defined from produced tracer concentrations, C(t), production rate, Qₚ(t), and injected tracer amount, M, as

$$E ( t ) = C ( t ) \cdot {Q}_{p} ( t ) / M \qquad ( 1 )$$

The unit of E is the inverse of the time unit. If a system has one injector and multiple producers j with production rates Qⱼ, we can define residence time distributions between each injector and producer j as

$${E}_{j} ( t ) = {C}_{j} ( t ) \cdot {Q}_{j} ( t ) / M \qquad ( 2 )$$

In a closed system the normalization by injected tracer amount ensures that

$${\sum }_{j} {\int }_{- ∞}^{∞} {E}_{j} ( t ) d t = 1 \qquad ( 3 )$$

where the sum is over all producers.

Important information about the geometry and flow in a system can be obtained from the moments of the residence time distribution. As an example, the zero and first order moments:

$${m}_{0 , j} = {\int }_{- ∞}^{∞} {E}_{j} ( t ) dt and {m}_{1 j} = {\int }_{- ∞}^{∞} t \cdot {E}_{j} ( t ) d t \qquad ( 4 )$$

represent respectively the relative amount of tracer produced in production well j, and the average residence time for the tracers between the injection well and producer j. In practical situations the zero order moment quantifies the significance of a fluid connection and the multiplication of the first order moment to the injection rate quantifies the volume explored by the traced fluid. In cases where produced fluid is re-injected, any contribution in tracer curves due to re-injection must be accounted for prior to making assessments due from the residence time distribution. This can be done in a systematic and unambiguous manner, using de-convolution. The residence time distribution at the outlet can be written as the convolution of the input signal and the injector-producer well-pair's residence time distribution function:

$${E}_{o u t} ( t ) = {\int }_{- ∞}^{t} {E}_{i n} ( t - {t}^{′} ) E ( {t}^{′} ) {dt}^{′} \qquad ( 5 )$$

If tracer is re-injected with a normalized re-injection concentration denoted by Eᵣ(t), the function that describes the total injected tracer is given by Eᵢₙ=δ(t)+Eᵣ(t) for t>0, where the initial tracer pulse injection is represented by the Dirac distribution. Setting this into Equation 5 and using the definition of a Dirac distribution and the commutative property of convolution integrals we find:

$$E ( t ) = {E}_{o u t} ( t ) - {\int }_{0}^{t} {E}_{r} ( {t}^{′} ) E ( t - {t}^{′} ) {dt}^{′} \qquad ( 6 )$$

This result states that at time t, the “true” tracer distribution from the delta pulse injection without re-injection, is given by the observed distribution Eₒᵤₜ(t), subtracted the integral up to time t of the “true” distribution and the known re-injection tracer distribution. E(t) may be calculated from known quantities. In practical implementations, the integral in Equation. 6 can be evaluated using numerical integration, e.g. using a trapezoid rule.

In many practical situations several producers may exist for each injector. The generalized result for one injector and several producers is given for each producer j as

$${E}_{j} ( t ) = {E}_{j , o u t} ( t ) - {\int }_{0}^{t} {E}_{r} ( {t}^{′} ) {E}_{j} ( t - {t}^{′} ) {dt}^{′} \qquad ( 7 )$$

where Eᵣ(t)=Cᵣ(t)Qₗ(t)/M is the distribution function for the reinjected tracer, Qₗ is the flowrate in the injector and M is the injected tracer amount, and where E_{j,out}(t)=Cⱼ(t)Qⱼ(t)/M denotes the distribution corresponding to measured concentration in well j. Eⱼ(t) is the corrected distribution we are looking for, related to concentration by Eⱼ(t)=Cⱼ(t)Qⱼ(t)/M.

In the general case injections into different injector wells may be performed at different times. The analysis above then needs to consider different times for a Dirac pulse. Practical situations may be further complicated by introducing multiple injectors. The analysis may require implementation and application of numerical computer models that can handle tracer transport. Use of these computations to deconvolve the tracer signals observed in the production wells require that information concerning the injection and production concentration signals as function of time are recorded.

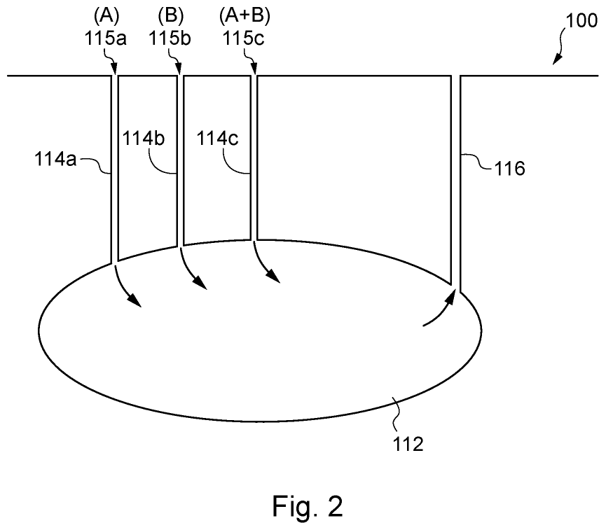

FIG. 2 is a simplified schematic of a geothermal system 100. The system 100 is similar to the geothermal system 10 described in FIG. 1 and will be understood from the description of FIG. 1 above. However, the system 100 described in FIG. 2 comprises three injection wells 114a, 114b and 114c and one producer well 116 in communication with geothermal reservoir 112. The power plant and injection apparatus have been removed for clarity.

In this example two tracers are used to monitor fluid from at least three possible injection sources. This mode of using several individual tracers and combining them to identify more sources than available tracer pose an alternative to using single unique tags to each of the sources of fluids, hence it can solve a problem of requiring a unique tracer for each source.

In this example fluid 115a injected into injection well 114a is labelled with tracer A. Fluid 115b injected into injection well 114b is labelled with tracer B. Fluid 115c injected into injection well 114c is labelled with equal levels of tracer A and tracer B. As it will be understood from the FIG. 1, the injected fluid passes through the reservoir and it is produced in the production well 116.

The method may comprise establishing a tracer concentration curve. The curve may be integrated to give the mass of tracer produced. The mass of tracer produced will be a certain fraction of the mass of tracer injected. The same fraction will be the fraction of fluid injected from the particular source and the volume of fluid produced.

If the tracer is not detected then the produced fluid is from a different injection source or the injection fluid from the tracer injection sources has not been transported yet through the reservoir. If samples of produced fluid contain tracer A only then the source of the fluid is injection well 114a. If samples of produced fluid contain tracer B only then the source of the fluid is injection well 114b. If samples of produced fluid contain identical concentrations of tracer A and tracer B which remain at identical ratio over time then the source of the fluid is injection well 114c. Monitoring tracer concentrations over time and/or modelling may enable a determination of whether the produced fluid are from a combination of the sources. Monitoring tracer concentrations over time and/or modelling may enable a determination of the contribution of each injection source to the produced fluid. The model may be updated based upon measured and/or calculated data. The reservoir model may employ history matching. History matching may use historical parameter measurements compared to calculated data. The parameters of the model may be adjusted until a reasonable match is achieved between the measured and calculated data.

Assuming there are four possible tracers available denoted by letters A, B, C and D. Two tracers can then be selected and combined in six possible ways: AB, AC, AD, BC, BD and CD if we consider that combinations such as AB and BA are indistinguishable.

Generally, we can combine k out of n tracers available and distinguish N_{S} different sources according to the following equation:

$${N}_{s} = \frac{n !}{k ! ( n - k ) !} \qquad ( 8 )$$

An advantage of using such a combination of tracers is that the number of unique tracer combinations N_{S} is large, as long as k is not too close to n and k is moderately large. As example, if we have n=6 tracers available and want to combine two (k=2) e.g. we can distinguish N_{S}=15 sources. If n=8 and k=3 we can distinguish N_{S}=56 sources. It is also possible to vary the number k of tracers in the combination. This was demonstrated in the example in FIG. 2 where n=2 and where k=1 was used for sources 114a, 114b and k=2 was used for source 114c. The tracer type and tracer concentrations may be modelled over time to determine the sources of the tracer injection. The produced fluid may be sampled and analysed for tracer at regular intervals. Where multiple sources use the same tracer in different tracer combinations, the sampling data may be modelled to deconvolute the tracer response data. The method may use modelling to attribute the measured concentrations of each tracer to a tracer combination of each tracer source. A mass balance may be performed by reservoir simulator or similar software to determine the distribution or pathway of injected fluid from one or more sources in the reservoir at any given point in time. Embodiments of the monitoring system and method may measure and/or monitor conditions of a geothermal reservoir and control the rate of injection of fluid and/or rate of production of fluid from the reservoir to optimise and/or control energy production.

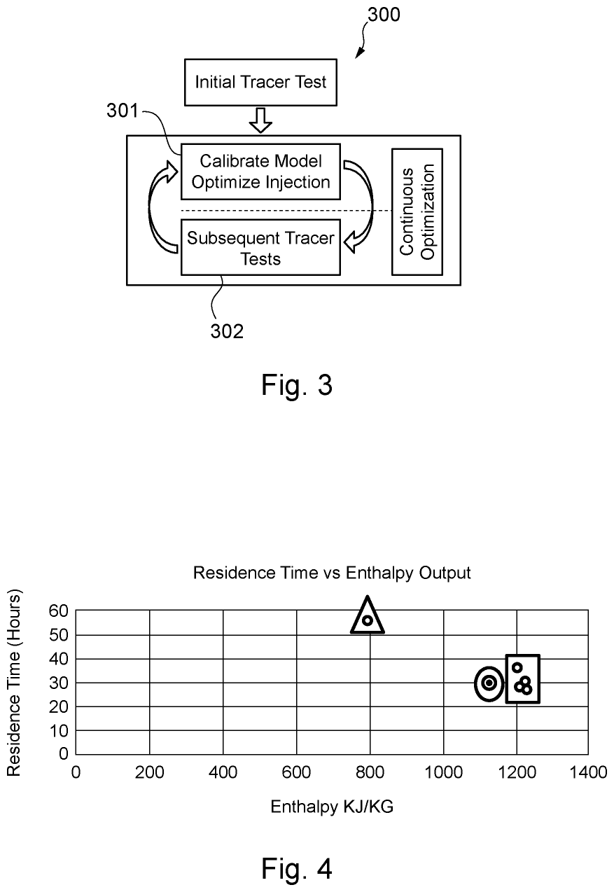

FIG. 3 is a flow diagram of the optimization of a model of the geothermal reservoir. Tracer data may be used to add resolution to the digital reservoir model and calibrate the predicted behaviour model based on future injection rates and volumes. An initial tracer test is carried out to confirm breakthrough from each unique injector well (injector contribution) to the reservoir and producer. The initial tracer test may establish a baseline residence time and enthalpy of the reservoir for an injector/producer pair. A residence time for injected water may calculated from each injector/producer pair. In this example the geothermal reservoir monitoring system comprises a tracer injection device and on-line detection device which may allow repeated or continuous tracer testing to calibrate the model (step 301) and optimise the energy extraction from the geothermal reservoir. This may include optimisation or improvement of the injection or production configuration selected from the group comprising injection rate, injection concentration, injection location, number of injection locations, injection time, injection well location, injection location in the injection well, injection frequency, injection volume, production rate, production location, production time, sampling time, sampling location, production frequency and/or production volume. Subsequent tracer tests (step 302) on the geothermal reservoir may further calibrate and/or confirm the model and allow for trend analysis. Subsequent tests may identify issues with the geothermal reservoir at an early stage. Event based tracer tests may also be used to identify or diagnose changes in the geothermal system such as a loss or reduction of enthalpy by injected water overcooling the reservoir. Regular or continuous tracer data may also be used to identify changes in residence time resulting in an early warning sign for cold water breakthrough or changes in connection between injectors, producers and/or the reservoir. The monitoring of flow through the geothermal reservoir using tracers may provide evidence of breakthrough and residence time. By monitoring the geothermal cycle with an injected tracer residence time can be identified and plotted against e.g. enthalpy output (KJ/KG) and/or flow rates which can help to optimize the injection strategy and prevent future loss of enthalpy.

FIG. 4 is a graph showing residence time plotted against enthalpy output (KJ/KG) for a number of tracer tests used to optimise the model and monitor energy extraction from the geothermal reservoir. FIG. 4 shows an initial tracer data point shown in the triangular box which provides evidence of communication and established a residence time and corresponding enthalpy output. Improvement of the operating strategy in combination with further tracer tests contributing to data shown in the square box in FIG. 4 will improve on the residence time and enthalpy output experience, optimising the model and the understanding of the total system performance and defining the operating envelope for the geothermal reservoir. This enables substantially consistent optimal residence times and enthalpy output forming the cluster of data points shown in FIG. 4. Finally, the tracer test shown as the tracer data point in the circle in FIG. 4 identified a change in the geothermal system resulting in a loss of enthalpy output which requires further investigation by the operator.

Monitoring the geothermal system over time may allow for continuous adjustment, optimization or improvement of the subterranean formation, which in this example is a geothermal system including identifying optimal injection rate, injection concentration, injection location, number of injection locations, injection time, injection well location, injection location in the injection well, injection frequency, injection volume, production rate, production location, production time, sampling time, sampling location, production frequency and/or production volume. This may facilitate an understanding of the total system performance. It may also enable identification of issues with the performance of the subterranean formation which is not possible via a single tracer test.

In the above example a geothermal reservoir is monitored. It will be appreciated that the system and methods may be used to monitor other subterranean formations including a hydrocarbon reservoir, a carbon capture storage reservoir, a subterranean hydrogen storage reservoir, a carbon dioxide storage reservoir, a water reservoir and/or a shale reservoir.

In the above example a continuous tracer injection is used. Continuous injection of tracers starting as injection fluid is injected may remove the lead time in tracer studies and may provide control on the mass balance in the field and may provide critical information during e.g. drilling new wells or side tracks. It may also provide information on the measurement of leakages and in mass balance calculations. If a continuous tracer injection is started at the same time as first injection into the subterrain, the origin of produced or leaked fluid from that subterrain may be determined in near real time. This could be especially useful in tracking of unknown paths to surface. This may be particularly useful to identify leaks from carbon capture reservoir where CO2 is injected for permanent storage. Additionally or alternatively the system may be configured for pulse tracer injections. In a pulse injection arrangement a known amount of tracer i.e. several kg to several hundred kg of tracer is injected into the injection well. The data obtained may be used to identify flow paths and flow directions and/or to calculate swept volumes, allocation factors and transport times.

In tracer injection systems the tracer may be released into the injection fluid in a number of ways. A topside pumping system may be configured to release tracer into the injection fluid with a constant tracer concentration and/or a short pulse of tracer. The release of tracer and the rate of release may be based on injection well flow rate feedback. The release of tracer into the injection fluid may be manual, automated and/or remotely operated. The release of tracer into the injection fluid may be on demand. Additionally or alternatively, tracer sources may be arranged such that it is in contact with injection fluid and the tracer leaches over time from the tracer source into the injection fluid.

The analysis of tracers in the samples may be a separate method to the collection of samples. Samples may be analysed at a time or jurisdiction which is separate and distinct from the sampling location and the collection of the samples. The analysis of measured tracer data may be a separate method to the collection or analysis of the samples. The tracer data may be analysed at a time or jurisdiction which is separate and distinct from the sampling location, collection of the samples and or measurement of tracer concentrations.

The invention may provide a system and method for monitoring a subterranean formation. The method may comprise injecting injection fluid into the at least one injection well wherein the at least one injection well is in fluid communication with the subterranean formation. The method may comprise injecting or releasing at least one tracer into the at least one injection well and taking at least one sample from fluid produced from the subterranean formation. The method may comprise measuring a concentration of the at least one tracer in the at least one sample and based on the measured concentration of the at least one tracer monitoring at least one characteristic of the subterranean formation. Throughout the specification, unless the context demands otherwise, the terms ‘comprise’ or ‘include’, or variations such as ‘comprises’ or ‘comprising’, ‘includes’ or ‘including’ will be understood to imply the inclusion of a stated integer or group of integers, but not the exclusion of any other integer or group of integers. Furthermore, relative terms such as “up”, “down”, “top”, “bottom”, “upper”, “lower”, “upward”, “downward”, “horizontal”, “vertical”, “extend”, “retract” and the like are used herein to indicate directions and locations as they apply to the appended drawings and will not be construed as limiting the invention and features thereof to particular arrangements or orientations.

The foregoing description of the invention has been presented for the purposes of illustration and description and is not intended to be exhaustive or to limit the invention to the precise form disclosed. The described embodiments were chosen and described in order to best explain the principles of the invention and its practical application to thereby enable others skilled in the art to best utilise the invention in various embodiments and with various modifications as are suited to the particular use contemplated. Therefore, further modifications or improvements may be incorporated without departing from the scope of the invention as defined by the appended claims.

1. A method for monitoring a subterranean formation, the method comprising:
injecting or releasing at least one tracer into at least one injection well, wherein the at least one injection well is in fluid communication with the subterranean formation;
injecting at least one injection fluid into the at least one injection well;
analysing at least one sample of fluid produced from the subterranean formation;
measuring a concentration of the at least one tracer in the at least one sample;
based on measured concentration of the at least one tracer optimising an injection configuration of the injection fluid and/or a production configuration of the produced fluid.

2. The method according to claim 1 wherein the subterranean formation is selected from the group consisting of a geothermal reservoir, a hydrocarbon reservoir, a carbon dioxide storage reservoir, a subterranean hydrogen storage reservoir and/or a water reservoir.

3. The method according to claim 1 comprising monitoring at least one characteristic of the subterranean formation based on the measured concentration of the at least one tracer.

4. The method according to claim 1, further comprising;
injecting or releasing the at least one tracer at a first time period, first injection fluid injection rate and/or first production rate to obtain a first tracer data set;
injecting or releasing the at least one tracer at a second time period, second injection fluid injection rate and/or second production rate to obtain a second tracer data set; and
comparing the first and second data sets to monitor at least one characteristic of the subterranean formation.

5. The method according to claim 4 comprising modelling the subterranean formation and calibrating or validating the model based on the first and second data sets.

6. The method according to claim 4 comprising identifying trends or changes in at least one characteristic of the subterranean formation based on a comparison of the first and second data sets.

7. The method according to claim 1 comprising injecting or releasing tracer into the injection fluid in response to a command signal, a manual command signal, an automated command signal, a pre-set programme, timer signal and/or trigger event.

8. The method according to claim 1 wherein the injection fluid and the at least one tracer are mixed before and/or during injection into the injection well.

9. The method according to claim 1 comprising adjusting and/or controlling a duration, concentration and/or frequency of an injection or release of tracer into the injection fluid.

10. The method according to claim 1 comprising injecting or releasing tracer continuously or as a pulse.

11. The method according to claim 1 wherein the sampling of the at least one sample of fluid is inline or online sampling.

12. The method according to claim 1 comprising measuring a concentration of the at least one tracer in the produced fluid in real time.

13. The method according to claim 1 comprising collecting the at least one sample for later analysis.

14. The method according to claim 1 comprising modelling the tracer concentration, transport time, injection volume, production volume, reservoir volume, injection flow rate and/or production rate in a model.

15. The method according to claim 1 comprising optimising and/or controlling the volume and/or rate of flow of fluid into, through and from the subterranean formation.

16. The method according to claim 1 comprising controlling and/or optimising the rate of injection to, and/or rate of producing fluid from, the subterranean formation based on measured concentration of the at least one tracer.

17. The method according to claim 1 comprising injecting injection fluid with a first tracer into a first injection well and injecting injection fluid with a second tracer into a second injection well.

18. The method according to claim 1 comprising releasing two or more tracers independently from one another or in synchrony with one another.

19. The method according to claim 1 wherein the at least one tracer is a tracer combination and/or a tracer combination ratio.

20. The method according to claim 1 comprising reinjecting produced fluid into the injection well and measuring and/or calculating a baseline of tracer in the reinjection fluid.

21. The method according to claim 1 wherein the subterranean formation is a geothermal reservoir the method comprising controlling an energy output of the geothermal reservoir by adjusting the injection rate of the injection fluid and/or adjusting a production rate of the produced fluid.

22. A system for monitoring a subterranean formation, the system comprising:
at least one tracer release device configured to inject or release at least one tracer into injection fluid;
at least one pump configured to pump or inject the injection fluid into at least one injection well;
at least one probe or sampling device;
wherein the at least one probe or sampling device is configured to measure the concentration of the at least one tracer in fluid produced from the subterranean formation-.

23. The system according to claim 22 wherein the at least one tracer release device is a tracer injection system configured to selectively release tracer into the injection fluid.

24. The system according to claim 22 wherein the at least one tracer release device is an automated tracer injection system configured to release tracer on demand.

25. The system according to claim 22 wherein the at least one probe is a sample collection probe, a detector probe and/or a real time detector probe.
